(19) United States
(12) Patent Application Publication
(10) Pub. No.: US 2026/0013722 A1
(43) Pub. Date: Jan. 15, 2026

(54) FUNDUS IMAGING SYSTEM

(71) Applicant: Medimaging Integrated Solution, Inc., Hsinchu (TW)

(72) Inventors: Chu-Ming Cheng, Hsinchu (TW); Yu-Tsung Lee, Hsinchu (TW); Keng-Han Chuang, Hsinchu (TW); CHING LUNG LAI, Hsinchu (TW)

(21) Appl. No.: 19/259,176

(22) Filed: Jul. 3, 2025

(30) Foreign Application Priority Data
Jul. 10, 2024 (TW) 113125736

(51) Int. Cl. A61B 3/12; A61B 3/00

(52) U.S. Cl. CPC A61B 3/1225; A61B 3/0058

(57) ABSTRACT

The invention discloses a fundus imaging system, which includes an optical path folding lens set, a virtual reality display, visible light fixation lamps, an illumination module, an image sensor, and a processor. The virtual reality display and the human eye are located on different sides of the optical path folding lens set. The virtual reality display forms images on the retina of the fundus of the human eye through the optical path folding lens set. The visible light fixation lamps are sequentially turned on to guide the eyeball of the human eye to rotate. When the visible light fixation lamp is turned on, the illumination module emits illumination light through the optical path folding lens set to illuminate the retina. The retina reflects the illumination light to form fundus light. The image sensor receives fundus light through the optical path folding lens set to form a sub-fundus image. The processor stitches all sub-fundus images into a full-fundus image and drives the virtual reality display to display the full fundus image.

### BACKGROUND OF THE INVENTION

### 1. Field of the Invention

The present invention relates to an imaging system, particularly to a fundus imaging system.

### 2. Description of the Prior Art

The solutions of virtual reality (VR) have gone through three main stages, including an aspherical lens stage, a Fresnel lens stage, and a folding optical path element stage. At present, the Fresnel lens is still the main optical solution of VR devices, and the folding optical path element is gradually adopted. The folding optical path not only is an important innovation of the optical system but also provides more space for the head-mounted VR devices. Therefore, it is expected to be the mainstream VR optical solution in the next few years.

Distinct from the direct optical path of the Fresnel lens, the folding optical path solution can greatly reduce the distance between the lens and the screen. It is a very important factor for a head-mounted device since the user should be unwilling to wear a thick and heavy device persistently and swing his head simultaneously. The solution of the folding optical path element can reduce the distance between the lens and the screen from 40-50 mm to 15-20 mm. If an ophthalmoscope is intended to be added into the limited space of a VR device without changing the original optical path design, the traditional design will lead to too large an ophthalmoscope size, which is unable to achieve a compact and lightweight feature required by a VR device. In the case shown in FIG. 1 where a working distance WD between an ophthalmoscope 10 and a human eye 12 is 40 mm and the field of view (FOV) is 200, it is estimated that the aperture CA should be at least 25 mm. However, such a size would cause a spatial problem.

The current VR device is unlikely to obtain retinal images. If the current retinal image capture device intends to acquire a complete retinal image, the human eye has to watch the lens inside the device straight, whereby the lens will form the retinal image on the image sensor behind the lens. In the current VR device, there are normally an optical imaging element and a display device before the human eye, and the optical imaging element projects the image of the display device onto the retina. Because of the abovementioned situation, the current VR device is unable to acquire fundus images. In the traditional design, adding an ophthalmoscope into a VR device will lead to too large a size, which does not meet the compactness and lightweight required by a VR device. If the volume of the ophthalmoscope is reduced, FOV would be sacrificed correspondingly.

Accordingly, the present invention proposes a fundus imaging system to overcome the conventional problems.

### SUMMARY OF THE INVENTION

The present invention provides a fundus imaging system, wherein the volume and weight of the overall system will not obviously increase.

In one embodiment of the present invention, the fundus imaging system comprises an optical path folding lens set, a virtual reality display, a plurality of visible light fixation lamps, an illumination module, an image sensor, and a processor. The optical path folding lens set includes an optical axis, a first side, and a second side opposite to the first side. The display face of the virtual reality display is faced toward a human eye. The virtual reality display and the human eye are respectively disposed at the first side and the second side of the optical path folding lens set and both positioned on the optical axis. The virtual reality display is used to project an imaging light through the optical path folding lens set onto the human eye to form an image on a retina of a fundus of the human eye. The visible light fixation lamps are disposed at the first side and turned on in sequence to guide rotation of the human eye. While the visible light fixation lamp is turned on, the illumination module emits an illumination light to light up the retina through the optical path folding lens set. The retina reflects the illumination light to form a fundus light. The image sensor is disposed at the first side, receiving the fundus light through the optical path folding lens set to form a sub-fundus image. The processor is coupled to the virtual reality display, the plurality of visible light fixation lamps, the illumination module and the image sensor. The processor turns on the plurality of visible light fixation lamps in sequence and drives the illumination module to emit the illumination light. The processor merges the sub-fundus images respectively corresponding to the plurality of visible light fixation lamps to form a full-fundus image and uses the virtual reality display to present the full-fundus image.

In one embodiment of the present invention, the visible light fixation lamps surround the image sensor.

In one embodiment of the present invention, the illumination light is a visible light or an invisible light.

In one embodiment of the present invention, the fundus imaging system further comprises a filter, which is disposed between the optical path folding lens set and the image sensor. The illumination light is an infrared light. The filter allows the infrared light to pass and reflects the visible light emitted by the visible light fixation lamps.

In one embodiment of the present invention, the fundus imaging system further comprises a network communication interface, which is coupled to the processor.

In one embodiment of the present invention, the network communication interface is in signal communication with a host computer. The host computer is coupled to a camera and an image display. The host computer downloads the full-fundus image and uses the image display to present the full-fundus image.

In one embodiment of the present invention, the optical path folding lens set is a pancake lens.

In one embodiment of the present invention, the image sensor is a complementary metal oxide semiconductor (CMOS) image sensor.

In one embodiment of the present invention, the illumination module is a light-emitting diode (LED) module.

In one embodiment of the present invention, the visible light fixation lamp is a light-emitting diode.

In summary, the fundus imaging system of the present invention carries an ophthalmoscope that comprises visible light fixation lamps, an illumination module, and an image sensor. The ophthalmoscope shares an optical path folding lens set and utilizes the characteristics of selective transmittance and selective reflection of the optical path folding lens set to fold the optical path of the ophthalmoscope and reduce the volume of the ophthalmoscope, whereby to realize miniaturization of the ophthalmoscope and make the volume and weight of the overall fundus imaging system not increase too much.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein

FIG. 1 is a diagram schematically showing the relative distance between the ophthalmoscope and the human eye in the conventional technology;

FIG. 2 is a diagram schematically showing a pancake lens according to one embodiment of the present invention;

FIG. 3 is a diagram schematically showing a fundus imaging system according a first embodiment of the present invention;

FIG. 4 is a diagram schematically showing the relative position of the visible light fixation lamps and the image sensor according to one embodiment of the present invention;

FIGS. 5a-5d are diagrams schematically showing the sub-fundus images respectively captured with different visible light fixation lamps according to one embodiment of the present invention;

FIG. 5e is a diagram schematically showing a full-fundus image according to one embodiment of the present invention;

FIG. 6 is a diagram schematically showing a fundus imaging system according to a second embodiment of the present invention;

FIG. 7 is a flowchart showing the operation process of a fundus imaging system according to one embodiment of the present invention; and

FIG. 8 is a diagram schematically showing the intercommunication between a fundus imaging system and a host computer according to one embodiment of the present invention.

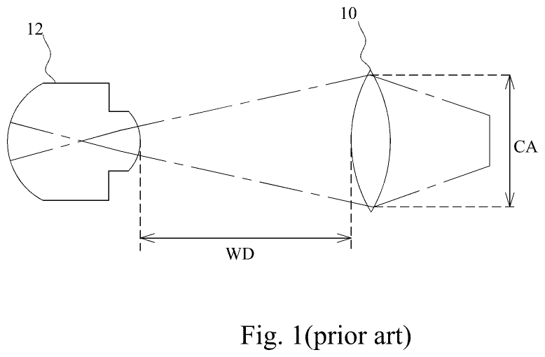

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

The embodiments of the present invention will be further demonstrated in details hereinafter in cooperation with the corresponding drawings. In the drawings and the specification, the same numerals represent the same or the like elements as much as possible. For simplicity and convenient labelling, the shapes and thicknesses of the elements may be exaggerated in the drawings. It is easily understood: the elements belonging to the conventional technologies and well known by the persons skilled in the art may be not particularly depicted in the drawings or described in the specification. Various modifications and variations made by the persons skilled in the art according to the contents of the present invention are to be included by the scope of the present invention.

While one element is described as “above one object”, it may indicate “the element is directly over the object” or “there is another thing between the element and the object”. While one element is described as “directly on one object”, it means “nothing exists between the element and the object”. The term “and/or” used in the specification and claims of the present invention refers to one or several of the listed items or any possible combination of the listed items, and the present invention includes these combinations.

Hereinafter, a description about “an embodiment” or “one embodiment” may be involved in a specified element, structure or characteristics of at least one embodiment. Therefore, the descriptions about “an embodiment” or “one embodiment” appearing in several positions of the text do not necessarily refer to the same embodiment. Besides, specified elements, structures or characteristics in one or more embodiments may be combined in an appropriate way.

The present invention is particularly described with the following embodiments. However, these embodiments are only to exemplify the present invention but not to limit the scope of the present invention. The persons skilled in the art can make modifications or variations of these embodiments without departing from the spirit and scope of the present invention. The scope of the present invention is based on the claims stated below. Unless it is particularly explained, the statement “a, one, or the/said element or component may imply the statement “one or at least one element or component”. While used in the specification and claims of the present invention, the singular noun, which is described by “a” “one”, “one piece of” or “the”, implies the plural form thereof unless the context indicates another condition clearly. In the specification or claims, “one thing is in one object” may indicate “the thing is in the object” and “the thing is on the object” unless the context explains it clearly. Each term used in the specification and claims normally has the ordinary meaning in the corresponding field the unless it is explained particularly. Some terms used in describing the present invention will be discussed in the specification to provide additional guidance for the practitioners. The terms used in any place of the specification, including the terms discussed additionally herein, will not be used to limit the scope and meaning of those terms but only used to exemplify those terms. Further, the scope of the present invention is not limited by the embodiments used in the specification.

The terms “comprising”, “including”, “having”, “containing” and “involving” mentioned in the specification and claims are open-ended terms and should be interpreted as “contain somethings and may further contain other things”. It is unnecessary for any embodiment or claim to realize all the targets or characteristics disclosed by the present invention. The abstract and title are only to assist in searching for the patent documents but not to limit the scope of the present invention.

It should be particularly explained: some conditional clauses or words, such as “can”, “could”, “might” and “may”, normally intend to express that the embodiment of the present invention has but is not limited to have a characteristics, element or step and that the characteristics, element or step is not necessarily indispensable in other embodiments.

Below is introduced a fundus imaging system, which carries an ophthalmoscope that comprises visible light fixation lamps, an illumination module, and an image sensor. The ophthalmoscope shares an optical path folding lens set and utilizes the characteristics of selective transmittance and selective reflection of the optical path folding lens set to fold the optical path of the ophthalmoscope and reduce the volume of the ophthalmoscope, whereby to realize miniaturization of the ophthalmoscope and make the volume and weight of the overall fundus imaging system not increase too much.

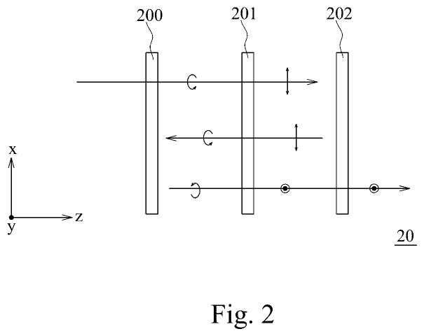

Refer to FIG. 2. FIG. 2 is a diagram schematically showing a pancake lens according to one embodiment of the present invention. The pancake lens can fold the optical path and thus can decrease weight and reduce the occupied space. Therefore, the pancake lens becomes the mainstream solution of the head-mounted display, particular the VR display. The principle of the pancake lens is to convert the polarization state of the polarized light and reflect the polarized light to fold the optical path and make the optical path exist between the half-mirror and a reflective polarizer. While the optical path is folded, the light passes the lenses between the half-mirror and the reflective polarizer three times, and the lens between the half-mirror and the reflective polarizer contributes diopter three times, whereby the pancake lens becomes compact and lightweight. As shown in FIG. 2, the pancake lens, which functions as the optical path folding lens set 20, includes a half-mirror 200, a quarter-wave plate 201, and a reflective polarizer 202. The half-mirror 200 reflects a half of the light, and the other half of the light passes through the half-mirror 200. Thus, the polarization state of the circularly polarized light is changed. For example, the half-mirror 200 reflects the left circularly polarized light to form the right circularly polarized light; or the half-mirror 200 reflects the right circularly polarized light to form the left circularly polarized light. The quarter-wave plate 201 converts the circularly polarized light into the linearly polarized light; or the quarter-wave plate 201 converts the linearly polarized light into the circularly polarized light. The reflective polarizer 202 reflects the first-direction linearly polarized light, and the second-direction linearly polarized light passes through the reflective polarizer 202, wherein the first direction is vertical to the second direction. In FIG. 2, both the solid-line arrow and the dotted-line arrow represent the directions of light propagation. The right circularly polarized light will pass through the half-mirror 200 and the quarter-wave plate 201 in sequence, converted into the x polarized light. The reflective polarizer 202 reflects the x polarized light. The quarter-wave plate 201 converts the x polarized light into the right circularly polarized light. The half-mirror reflects the right circularly polarized light to form the left circularly polarized light. The left circularly polarized light passes through the quarter-wave plate 201 to form a y polarized light. The y polarized light directly passes through the reflective polarizer 202. The pancake lens is used in the fundus imaging system of the present invention.

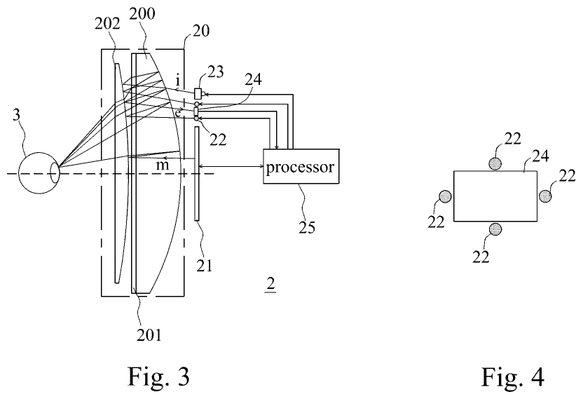

Refer to FIG. 3 and FIG. 4. FIG. 3 schematically shows a fundus imaging system according a first embodiment of the present invention. FIG. 4 schematically shows the relative position of the visible light fixation lamps and the image sensor according to one embodiment of the present invention. Below is introduced a fundus imaging system 2 of the present invention. The fundus imaging system 2 comprises an optical path folding lens set 20, a virtual reality display 21, a plurality of visible light fixation lamps 22 respectively disposed at different positions, an illumination module 23, an image sensor 24, and a processor 25. The visible light fixation lamps 22, the illumination module 23 and the image sensor 24 jointly form an ophthalmoscope. In some embodiments, the illumination module 23 may be but is not limited to be a light-emitting diode module. In some embodiments, the visible light fixation lamps 22 may be but are not limited to be light-emitting diodes. In some embodiments, the image sensor 24 may be but is not limited to be a complementary metal oxide semiconductor (CMOS) image sensor. The optical path folding lens set 20 includes an optical axis, which is expressed by a dotted line, a first side, and a second side opposite to the first side. The optical path folding lens set 20 reflects and transmits light to realize folding of the optical path. The optical path folding lens set 20 can fold the optical path of the ophthalmoscope and reduce the volume thereof, whereby to realize miniaturization of the ophthalmoscope. Thus, the volume and weight of the overall fundus imaging system 2 will not increase too much. The display face of the virtual reality display 21 is faced toward a human eye 3, wherein the virtual reality display 21 and the human eye 3 are respectively situated at the first side and the second side of the optical path folding lens set 20 and both located on the optical axis. The visible light fixation lamps 22, the illumination module 23, and the image sensor 24 are disposed on the first side of the optical path folding lens set 20. In some embodiments of the present invention, the visible light fixation lamps 22 surround the image sensor 24. The processor 25 is coupled to the virtual reality display 21, the visible light fixation lamps 22, the illumination module 23, and the image sensor 24.

The virtual reality display 21 projects an imaging light m through the optical path folding lens set 20 to the human eye 3 to form an image on the retina of the fundus. The visible light fixation lamps 22 turn on in sequence to guide the rotation of the eyeball of the human eye 3. The light emitted by the visible light fixation lamps 22 is reflected and transmitted by the optical path folding lens set 20 and then enters the eyeball to form an image. The visible light fixation lamps 22 function to guide the eyeball of the testee to rotate, whereby to align the eyeball to the illumination system and fundus imaging system of the fundus image capture system. Because the visible light fixation lamps 22 are disposed at off-axis positions, the head of the testee must be fixed during operation, and the turned-on visible light fixation lamp 22 is gazed at via merely rotating the eyeball. While the visible light fixation lamp 22 is turned on, the illumination light i, which is emitted by the illumination module 23, passes through the optical path folding lens set 20 to illuminate the retina. The illumination light i may be a visible light or an invisible infrared light. The retina reflects the illumination light i to form a fundus light e. The illumination module 23 may include optical lenses. The light, which is emitted by the illumination module 23, is reflected and transmitted by the optical path folding lens set 20, next focused on the pupil and then spread to illuminate the retina of the fundus. The illumination is to light up the fundus, whereby the light, which is reflected by the fundus, can be captured to obtain the image of the fundus. While the fundus is illuminated, the light, which reflected by the fundus, passes through the eyeball and then is reflected and transmitted by the optical path folding lens set 20. Finally, the light is focused on the image sensor 24 to form an image. In other words, the image sensor 24 receives the fundus light e through the optical path folding lens set 20 to form a sub-fundus image. While turning on the visible light fixation lamps 22 in sequence and driving the illumination module 23 to emit the illumination light I, the processor 25 merges the sub-fundus images, which are respectively corresponding to the plurality of visible light fixation lamp 22, into a full-fundus image and uses the virtual reality display 21 to present the full-fundus image.

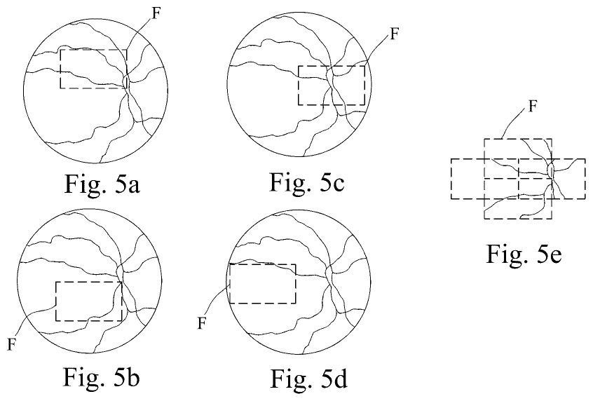

Refer to FIG. 4 and FIGS. 5a-5d. FIGS. 5a-5d schematically show the sub-fundus images respectively captured with different visible light fixation lamps according to one embodiment of the present invention. In order to increase the observation scope, different fixation lamps, which are disposed at different positions, are used to capture sub-fundus images from different angles. The sub-fundus images may be merged to form a large-scale full-fundus image. FIG. 5a schematically shows a sub-fundus image captured by the image sensor 24 with the visible light fixation lamp 22 disposed below the image sensor 24 being turned on, wherein the area enclosed by the dotted frame F is the location of the image sensor 24. FIG. 5b schematically shows a sub-fundus image captured by the image sensor 24 with the visible light fixation lamp 22 disposed above the image sensor 24 being turned on, wherein the area enclosed by the dotted frame F is the location of the image sensor 24. FIG. 5c schematically shows a sub-fundus image captured by the image sensor 24 with the visible light fixation lamp 22 disposed at the left side of the image sensor 24 being turned on, wherein the area enclosed by the dotted frame F is the location of the image sensor 24. FIG. 5d schematically shows a sub-fundus image captured by the image sensor 24 with the visible light fixation lamp 22 disposed at the right side of the image sensor 24 being turned on, wherein the area enclosed by the dotted frame F is the location of the image sensor 24. FIG. 5e schematically shows a full-fundus image according to one embodiment of the present invention, wherein FIGS. 5a-5d are merged to form FIG. 5e.

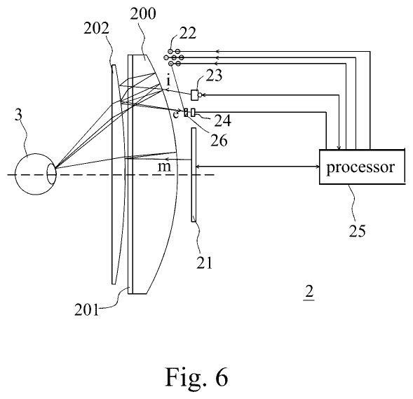

FIG. 6 schematically shows a fundus imaging system according to a second embodiment of the present invention. The second embodiment is different from the first embodiment in that the visible light fixation lamps 22 are disposed diagonally above the illumination module 23 and that the fundus imaging system 2 of the second embodiment further comprises a filter 26, which is disposed between the optical path folding lens set 20 and the image sensor 24. The illumination light i is an infrared light. The filter 26 allows the infrared light to pass and reflects the visible light emitted by the visible light fixation lamps 22. The visible light emitted by the visible light fixation lamps 22 will hit the filter 26 and will be reflected by the filter 26. Next, the visible light is transmitted and reflected by the optical path folding lens set 20 and then reaches the fundus. The user gazes at the visible light fixation lamps disposed at different positions in sequence, whereby the image sensor 24 may capture the fundus images of different positions. The other technical characteristics of the second embodiment are identical to those of the first embodiment and will not repeat herein.

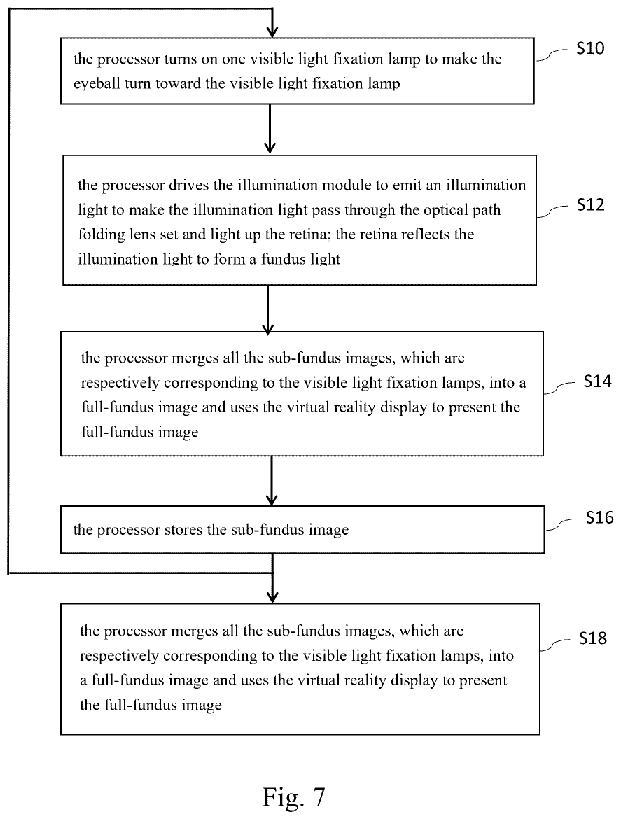

FIG. 7 is a flowchart showing the operation process of a fundus imaging system according to one embodiment of the present invention. Refer to FIG. 7 and FIG. 3. In Step S10, the processor 25 turns on one visible light fixation lamp 22 to make the eyeball turn toward the visible light fixation lamp 22. In Step S12, the processor 25 drives the illumination module 23 to emit an illumination light i to make the illumination light i pass through the optical path folding lens set 20 and light up the retina; the retina reflects the illumination light i to form a fundus light e. In Step S14, the image sensor 24 receives the fundus light e through the optical path folding lens set 20 to form a sub-fundus image. In Step S16, the processor 25 stores the sub-fundus image. After Step S16, the process returns to Step S10 until all the visible light fixation lamps 22 were once lighted up in turns. If there are four visible light fixation lamps 22, the process from Step S10 to Step S14 will be repeated four times. As long as all the visible light fixation lamps 22 have been lighted up once, Step S18 is performed after Step S16. In Step S18, the processor 25 merges all the sub-fundus images, which are respectively corresponding to the visible light fixation lamps 22, into a full-fundus image and uses the virtual reality display 21 to present the full-fundus image.

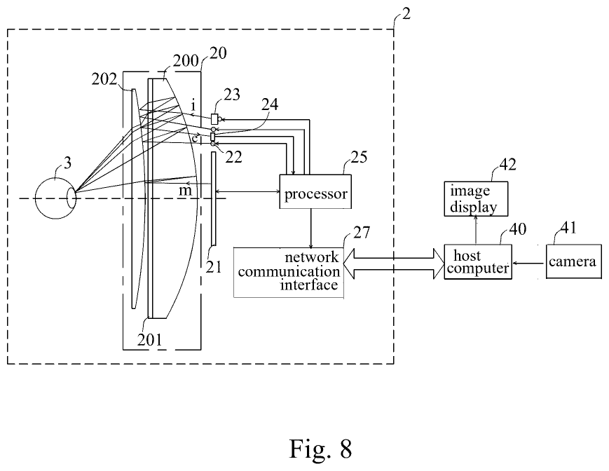

FIG. 8 is a diagram schematically showing the intercommunication between a fundus imaging system and a host computer according to one embodiment of the present invention. In comparison with the fundus imaging system shown in FIG. 3, the fundus imaging system shown in FIG. 8 further comprises a network communication interface 27, which is coupled to the processor 25. The network communication interface 27 may communicate with a host computer 40 through network signals. The host computer 40 has a built-in network communication interface. The host computer 40 is coupled to a camera 41 and an image display 42. The host computer 40 may download the full-fundus image and use the image display 42 to present the full-fundus image. In other words, the medical personnel may undertake bidirectional remote video medicine through the image display 42, and the user may view how the medical personnel diagnose the fundus through the virtual reality display 21.

According to the abovementioned embodiments, the fundus imaging system of the present invention carriers an ophthalmoscope, which is formed by visible light fixation lamps, an illumination module and an image sensor. The ophthalmoscope shares an optical path folding lens set and utilizes the characteristics of selective transmittance and selective reflection of the optical path folding lens set to fold the optical path of the ophthalmoscope and reduce the volume of the ophthalmoscope, whereby to realize miniaturization of the ophthalmoscope and make the volume and weight of the overall fundus imaging system not increase too much.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

What is claimed is:

1. A fundus imaging system comprising
an optical path folding lens set, including an optical axis, a first side, and a second side opposite to the first side;
a virtual reality display, whose display face is faced toward a human eye, wherein the virtual reality display and the human eye are respectively disposed at the first side and the second side of the optical path folding lens set and both positioned on the optical axis; the virtual reality display is used to project an imaging light through the optical path folding lens set onto the human eye to form an image on a retina of a fundus of the human eye;
a plurality of visible light fixation lamps, disposed at the first side, and turned on in sequence to guide rotation of the human eye;
an illumination module, disposed at the first side, wherein while the visible light fixation lamp is turned on, the illumination module emits an illumination light to light up the retina through the optical path folding lens set; the retina reflects the illumination light to form a fundus light;
an image sensor, disposed at the first side, and receiving the fundus light through the optical path folding lens set to form a sub-fundus image; and
a processor, coupled to the virtual reality display, the plurality of visible light fixation lamps, the illumination module and the image sensor, turning on the plurality of visible light fixation lamps in sequence, driving the illumination module to emit the illumination light, merging the sub-fundus images respectively corresponding to the plurality of visible light fixation lamps to form a full-fundus image, and using the virtual reality display to present the full-fundus image.

2. The fundus imaging system according to claim 1, wherein the plurality of visible light fixation lamps surrounds the image sensor.

3. The fundus imaging system according to claim 1, wherein the illumination light is a visible light or an invisible light.

4. The fundus imaging system according to claim 1 further comprising a filter, which is disposed between the optical path folding lens set and the image sensor, wherein the illumination light is an infrared light; the filter allows the infrared light to pass and reflects the visible light emitted by the visible light fixation lamps.

5. The fundus imaging system according to claim 1 further comprising a network communication interface, which is coupled to the processor.

6. The fundus imaging system according to claim 5, wherein the network communication interface is in signal communication with a host computer; the host computer is coupled to a camera and an image display; the host computer downloads the full-fundus image and uses the image display to present the full-fundus image.

7. The fundus imaging system according to claim 1, wherein the optical path folding lens set is a pancake lens.

8. The fundus imaging system according to claim 1, wherein the image sensor is a complementary metal oxide semiconductor (CMOS) image sensor.

9. The fundus imaging system according to claim 1, wherein the illumination module is a light-emitting diode (LED) module.

10. The fundus imaging system according to claim 1, wherein the plurality of visible light fixation lamps is light-emitting diodes.
